(19) United States
(12) Patent Application Publication
(10) Pub. No.: US 2024/0090769 A1
(43) Pub. Date: Mar. 21, 2024

(54) SPECTROMETRY SYSTEMS, METHODS, AND APPLICATIONS

(71) Applicant: The Brigham and Women's Hospital, Inc., Boston, MA (US)

(72) Inventors: William B. GORMLEY, Boston, MA (US); Paola Calvachi Prieto, Boston, MA (US)

(21) Appl. No.: 18/039,777

(22) PCT Filed: Dec. 1, 2021
(86) PCT No.: PCT/US21/61385

§ 371 Date: Jun. 1, 2023

Related U.S. Application Data
(60) Provisional application No. 63/120,025, filed on Dec. 1, 2020.

(51) Int. Cl. A61B 5/00; A61B 5/145; A61B 5/1459

(52) U.S. Cl. CPC A61B 5/0075; A61B 5/0022; A61B 5/14514; A61B 5/1459; A61B 5/6852; A61B 5/7267

(57) ABSTRACT

An indwelling catheter surveillance system which can detect and distinguish a clean catheter system from one with bacterial colonization and from one with bacterial infection. This is done using a micro-spectroscopy system placed on the outside of an indwelling catheter's drainage tube with analysis being facilitated using machine learning algorithms. This system is based on the ability to leverage the analysis of bacteria and biomarkers in liquid bio samples at the patient's bedside, in real time to deliver a mobile, continuous, point of care, disposable and cost-effective solution. This represents a feasible and scalable system for resolving the problem of infections in indwelling catheter systems.

### CROSS-REFERENCE TO RELATED APPLICATIONS

This application claims the benefit of U.S. Provisional Patent Application No. 63/120,025 filed Dec. 1, 2020, the disclosure of which, as well as the references cited therein, is hereby incorporated by reference.

### STATEMENT OF FEDERALLY SPONSORED RESEARCH OR DEVELOPMENT

N/A.

### BACKGROUND OF THE INVENTION

Healthcare acquired infections are a major problem. These include infections both in hospitals or healthcare facilities as well as infections in the home setting related to patients managing their own medical related procedures. Prevalence of hospital acquired infections (HAIs) in the United States alone is measured at 1.7 million infections per year with a human cost that translates into 99,000 deaths. The financial toll in the United States is measured somewhere near $45 billion a year. It is clear then, that these infections pose a serious human and financial cost to the American healthcare system. Human and financial costs associated with infections related to patient delivered, home medical care procedures are less well quantified but are estimated to total in the billions of dollars as well. This of course, is not a problem restricted to the United States and is seen throughout the developed and developing world.

HAIs are exemplified by catheter associated urinary tract infections (CAUTIs) which represent one of the most common and most serious forms. They represent approximately 32% of all HAIs affecting over 2.5 million patients a year with a high level of morbidity and a mortality rate of approximately 13,000 deaths per year. It is estimated that each of these infections has an additional cost of $13,731 per case. In aggregate the cost to the United States healthcare system for catheter associated urinary tract infection approaches $7.7 billion per year. Given this, it is clear that the financial and human cost of this CAUTIs is significant.

Infections related to patient delivered, home medical care procedures are exemplified by those related to patients utilizing peritoneal dialysis for management of end stage renal disease (ESRD). Peritoneal dialysis is a method in which the peritoneum, the lining of the abdominal cavity, is used as a natural filtration device. This requires an indwelling peritoneal catheter through which daily exchanges of a cleansing fluid called dialysate, are performed in cycles to accomplish the goal of fluid management and toxin removal that the failed kidneys can no longer perform. Peritoneal dialysis seems to be associated with 48% lower mortality than hemodialysis over the first 2 years of dialysis therapy independent of modality switches or differential transplantation rates and is widely viewed as a preferable solution over hemodialysis for patients with ESRD. Despite this fact, hemodialysis remains the dominant modality for management of ESRD because of the risk that abdominal infection, known as peritonitis, presents to patients using peritoneal dialysis. PD-associated peritonitis is the direct or major contributing cause of death in >15% of patients on PD.

In both of these examples, and others in which body cavities are instrumented with indwelling catheters, the need for monitoring and early detection of infection is critical if the human and financial costs of these infections are to be avoided.

In addition to the identification of HAI, access to biosamples through existing indwelling patient catheters for the purpose of in vivo and continuous analysis makes the identification of biomarkers encountered in any human fluid including, but not limited to urine peritoneal fluid, wound drainage, enteral content, etc., of significant value to early detection of disease and guidance of therapy.

### SUMMARY OF THE INVENTION

Thus, there is a need to monitor for HAIs, and in particular for CAUTIs, and for infections related to patient delivered, home medical care procedures.

Various embodiments of the disclosed invention provide for and expand upon methods, apparatus, and systems for continuous, real time, on-catheter, on-patient, bacterial colonization and bacterial infection product detection. In the present description we demonstrate a real-time system for continuous screening, detection, and alerting of clinical personnel as to the state of a patient's liquid bio sample system relating to infection and to the identification of biomarkers associated with the functional status of multiple human systems including the cardiac, renal, respiratory, neurologic, endocrine, and immune systems.

The present invention relates a method of screening a sample for the presence of one or more compounds of interest. The method uses a NIR spectrometer to analyze the fluid. Additionally, different Machine Learning algorithms were created in order to classify and identify different types of bacteria within the fluid and some other bioproducts.

The current embodiment includes a system for continuous liquid bio sample screening without interrupting the flow of liquid bio sample through the use of a specific device design which will allow for liquid bio sample to be without flow long enough for testing, without the interruption of liquid bio sample flow without the application of external blockers.

In various embodiments, a biofluid monitoring apparatus may be provided. The apparatus may include: a spectrometer disposed within a housing, the spectrometer including: a light source to illuminate a sample within a catheter tubing, a detector to detect light returned from the sample, a status signal indicator to provide patient status based on the sample in the catheter tubing, and a controller in communication with the light source, the detector, and the status signal indicator to collect and process data based on the light returned from the sample to determine a patient status and indicate the patient status using the status indicator, wherein the housing is configured to attach at a low point in the catheter tubing such that the sample accumulates in the low point, and wherein the light source and the detector are directed towards the low point to obtain the data from the sample.

In some embodiments, the spectrometer may further include a power supply. In certain embodiments, the power supply may include a battery.

In various embodiments, the housing may include a slot into which the catheter tubing is inserted such that a portion of the catheter tubing is adjacent to the spectrometer.

In particular embodiments, the spectrometer may further include a collimator to focus light from the light source into the sample. In some embodiments, the collimator may include a lens.

In certain embodiments, the spectrometer may further include a monochromator to divide the light from the light source into a plurality of constituent wavelengths. In various embodiments, the monochromator may include a prism. In particular embodiments, the spectrometer may further include a wavelength selector to select a particular wavelength to direct to the sample, where the particular wavelength may be selected based on at least one of a bacterial strain or a bacterial product to be identified. In some embodiments, the wavelength selector may include a slit.

In various embodiments, the detector may include a photocell to record one or more wavelengths of light returned from the sample based on the illumination of the sample. In some embodiments, the light returned from the sample measured by the detector may include absorbance information.

In certain embodiments, the spectrometer may further include a communication module to transmit information from the spectrometer. In some embodiments, the communication module may include a radio communication device including at least one of a. Bluetooth device, a cellular service device, or a WiFi device for performing wireless transmission. In various embodiments, the radio communication device including at least one of a Bluetooth device, cellular service device, or Win device may perform wireless transmission to a computing platform including at least one of an electronic health record or a mobile computing device. In particular embodiments, the mobile computing device may include at least one of a cell phone, a smart phone, a pager, or a telephone. In some embodiments, the information from the spectrometer may be transmitted as at least one of a text message, an audio message, an email, or a data file.

In particular embodiments, the controller may determine the patient status using one or more machine learning algorithms specifically trained for the apparatus. In some embodiments, the one or more machine learning algorithms may identify one or more biomarkers indicative of a functional status of a bodily system of the patient. In various embodiments, the bodily system of the patient may include at least one of a cardiac system, a respiratory system, a renal system, a neurologic system, an endocrine system, or an immune system. In certain embodiments, the one or more machine learning algorithms may identify at least one condition comprising at least one of: a bacterial colony count, a bacterial colony type, or a bacterial infection by-product. In some embodiments, the patient status may be determined based on the identified at least one condition.

In various embodiments, the status indicator may be configured to indicate at least one of a plurality of states of the patient status. In certain embodiments, the states of the patient status may include at least one of: no bacteria or infection in the sample, bacterial colonization but no infection in the sample, or bacteria and infection in the sample. In some embodiments, the status indicator may indicate the patient status using at least one light coupled to the housing.

In particular embodiments, the controller may determine the patient status using one or more machine learning algorithms specifically trained for the apparatus. In certain embodiments, the one or more machine learning algorithms may identify biomarkers related to the functional status of the patient's cardiac system. In some embodiments, the patient status may be determined based on the identified at least one condition.

In particular embodiments, the controller may determine the patient status using one or more machine learning algorithms specifically trained for the apparatus. In certain embodiments, the one or more machine learning algorithms may identify biomarkers related to the functional status of the patient's respiratory system. In some embodiments, the patient status may be determined based on the identified at least one condition.

In particular embodiments, the controller may determine the patient status using one or more machine learning algorithms specifically trained for the apparatus. In certain embodiments, the one or more machine learning algorithms may identify biomarkers related to the functional status of the patient's renal system. In some embodiments, the patient status may be determined based on the identified at least one condition.

In particular embodiments, the controller may determine the patient status using one or more machine learning algorithms specifically trained for the apparatus. In certain embodiments, the one or more machine learning algorithms may identify biomarkers related to the functional status of the patient's neurologic system. In some embodiments, the patient status may be determined based on the identified at least one condition.

In particular embodiments, the controller may determine the patient status using one or more machine learning algorithms specifically trained for the apparatus. In certain embodiments, the one or more machine learning algorithms may identify biomarkers related to the functional status of the patient's endocrine system. In some embodiments, the patient status may be determined based on the identified at least one condition.

In particular embodiments, the controller may determine the patient status using one or more machine learning algorithms specifically trained for the apparatus. In certain embodiments, the one or more machine learning algorithms may identify biomarkers related to the functional status of the patient's immune system. In some embodiments, the patient status may be determined based on the identified at least one condition.

In particular embodiments, the low point in the catheter tubing may include a bend in the catheter tubing. In various embodiments, the housing may include a curved face and the bend in the catheter tubing may be located adjacent to the curved face of the housing.

In some embodiments, the apparatus may further include a load cell sensor coupled to the housing, where the load cell sensor may be coupled to a biofluid collection container fluidly coupled to the catheter tubing. The controller may be coupled to the load cell sensor and may be configured to: obtain data from the load cell sensor, calculate a weight change of the biofluid collection container based on the data Obtained from the load cell sensor, and determine a flow rate of the sample into the biofluid collection contained based on the calculated weight change.

In various embodiments, biofluid monitoring method may be provided. The method may include: providing a spectrometer disposed within a housing, where the spectrometer may include: a light source to illuminate a sample within a catheter tubing, a detector to detect light returned from the sample, a status signal indicator to provide patient status based on the sample in the catheter tubing, and a controller in communication with the light source, the detector, and the status signal indicator; collecting and processing, using the controller, data based on the light returned from the sample; determining, using the controller and based on collecting and processing the data, a patient status; and indicating, using the controller, the patient status using the status indicator, wherein the housing may be configured to attach at a low point in the catheter tubing such that the sample accumulates in the low point, and wherein the light source and the detector may be directed towards the low point to obtain the data from the sample.

In some embodiments, the spectrometer may further include a power supply. In certain embodiments, the power supply may include a battery.

In certain embodiments, the housing may include a slot into which the catheter tubing may be inserted such that a portion of the catheter tubing is adjacent to the spectrometer.

In particular embodiments, the spectrometer may further include a collimator and the method may further include focusing light from the light source into the sample using the collimator. In some embodiments, the collimator may include a lens.

In various embodiments, the spectrometer may further include a monochromator and the method may further include dividing the light from the light source into a plurality of constituent wavelengths using the monochromator. In some embodiments, the monochromator may include a prism. In certain embodiments, the spectrometer may further include a wavelength selector and the method may further include selecting a particular wavelength to direct to the sample using the wavelength selector, wherein the particular wavelength is selected based on at least one of a bacterial strain or a bacterial product to be identified. In some embodiments, the wavelength selector may include a slit.

In particular embodiments, the detector may include a photocell and the method may further include recording one or more wavelengths of light returned from the sample based on the illumination of the sample using the photocell. In some embodiments, the light returned from the sample measured by the detector may include absorbance information.

In certain embodiments, the spectrometer may further include a communication module and the method may further include transmitting information from the spectrometer using the communication module. In some embodiments, the communication module may include a radio communication device including at least one of a Bluetooth device, a cellular service device, or a WiFi device, wherein transmitting information from the spectrometer using the communication module may further include transmitting information wirelessly from the spectrometer using the radio communication device including at least one of a Bluetooth device, cellular service device, or WiFi device. In various embodiments, the radio communication device including at least one of a Bluetooth, cellular service, or WiFi device may perform wireless transmission to a computing platform including at least one of an electronic health record or a mobile computing device. In particular embodiments, the mobile computing device may include at least one of a cell phone, a smart phone, a pager, or a telephone. In some embodiments, the information from the spectrometer may be transmitted as at least one of a text message, an audio message, an email, or a data file.

In some embodiments, determining the patient status may further include determining the patient status using one or more machine learning algorithms specifically trained for the apparatus. In certain embodiments, determining the patient status using one or more machine learning algorithms specifically trained for the apparatus may further include identifying one or more biomarkers indicative of a functional status of a bodily system of the patient using the one or more machine learning algorithms. In some embodiments, the bodily system of the patient comprises at least one of a cardiac system, a respiratory system, a renal system, a neurologic system, an endocrine system, or an immune system. In various embodiments, the one or more machine learning algorithms may identify at least one condition including at least one of: a bacterial colony count, a bacterial colony type, or a bacterial infection by-product. In particular embodiments, determining the patient status using one or more machine learning algorithms may further include determining the patient status based on the identified at least one condition.

In certain embodiments, indicating the patient status using the status indicator may further include indicating at least one of a plurality of states of the patient status. In some embodiments, the states of the patient status may include at least one of: no bacteria or infection in the sample, bacterial colonization but no infection in the sample, or bacteria and infection in the sample. In particular embodiments, indicating the patient status using the status indicator may further include indicating the patient status using at least one light coupled to the housing.

In some embodiments, the low point in the catheter tubing may include a bend in the catheter tubing. In certain embodiments, the housing may include a curved face, and the bend in the catheter tubing may be located adjacent to the curved face of the housing.

In various embodiments, the device may calculate an approximation of flow rate of a biofluid that passes through the indwelling catheters and therefore calculate an actual measurement of amount of biofluid coming from the patient at any given time. In certain embodiments, flow rate may be calculated through use of a measurement of changing weight in a biofluid repository (e.g. a biofluid collection bag) over time. In particular embodiments, flow rate may be calculated on a continuous basis as a measure of weight change and may be reported to the user one or more communication mechanism of the device. In various embodiments, the calculated flow rate may be based on the following formula: δ weight/δ time. In some embodiments, an algorithm may utilize this data to calculate a volume over time calculation to yield an approximation of flow rate over time. This data may be reported to the user continuously via one or more communications mechanisms of the device.

Accordingly, in some embodiments the housing, may include a load cell sensor coupled thereto, where the load cell sensor may be coupled to a biofluid collection container fluidly coupled to the catheter tubing, and the method may further include: obtaining data from the load cell sensor, calculating a weight change of the biofluid collection container based on obtaining the data from the load cell sensor, and determining a flow rate of the sample into the biofluid collection contained based on calculating the weight change.

### BRIEF DESCRIPTION OF THE DRAWINGS

FIG. 1 shows an on-catheter device design with or without block. This shows how, with the design of the device and its housing structure, the catheter can be shaped in such a way that continuous readings of the column of liquid bio sample can be carried out with or without a catheter blockage device.

FIG. 2 shows an on-catheter device design in a close up—lateral view. This demonstrates how the spectrometer on the device interacts with the catheter and the liquid bio sample stream inside of it.

FIGS. 3A, 3B, and 3C show detailed views of another on-catheter design including the micro-spectrometer with its catheter mount.

FIG. 4 shows the entire clinical setting with the urinary catheter in its natural position on a patient, including the incorporated urinary spectrometry screening, detection and alerting device (sensor C). This depicts an embodiment of a method for how continuous, real time, on-catheter measurements may be performed.

FIG. 5 shows a diagram of the on-catheter device in which it is broken down into its component parts which include power, a micro-spectrometer, a light source, a collimator (lens), a monochromator (prism), a wavelength selector (slit), the patient's liquid bio sample, the detector (photocell), a Bluetooth device for transmission to a computing platform (e.g. to an electronic health record or a mobile computing device where clinicians can see results), and an on-device signal so that clinicians can visualize results without leaving the patient's setting.

FIG. 6 provides a demonstration of how the device's spectrum of emitted light is transmitted to the computational platform for analysis by machine learning algorithms to return the molecular signal of the sample including 1) the bacterial colony count, 2) the bacterial colony type, and 3) bacterial infection by-products identifiable in the sample.

FIG. 7 shows a clinical interface in accordance with certain embodiments of the invention.

FIG. 8 shows a spectrometry-analytics workflow in accordance with certain embodiments of the invention.

FIG. 9 shows raw and processed data which reflect the absorbance spectra plots for E. coli scanned with a micro-NIR spectrometer in the wavelength range of 740 nm-1100 nm, with the x-axis being wavelength (urn) and the y-axis being absorbance (normalized from 0.0 to 1.0).

FIG. 10 shows three-dimensional principal component analysis of the data obtained in FIG. 9.

FIGS. 11A and 11B show the results of both model types developed, classification models and regression models: 1) Random Forest; 2) Extreme Gradient Boosting; 3) Linear Regression, fits a linear model to minimize the residual sum of squares between the observed targets in the dataset, and the targets predicted by the linear approximation; 4) Elastic Net, which is a combination between lasso and ridge regression and 5) Lasso and Elastic-Net Regularized Generalized Linear Models that fits a generalized linear model via penalized maximum likelihood. The regularization path is computed for the lasso or elastic network penalty at a grid of values for the regularization parameter lambda. R² also called coefficient of determination, is a regression score function, Best possible R² score is 1.0 and it can be negative. A constant model that always predicts the expected value of y, disregarding the input features, would get a R² score of 0.0. RMSE measures the differences between values predicted by a model and the values observed. MAE is the sum of absolute differences between our target and predicted variables, it measures the average magnitude of errors in a set of predictions, without considering their directions. FIG. 11A shows classification models (Median ROC scores) including dichotomous analyses for the five classification models: 1) Logistic Regression, 2) Random forests (RF); 3) Gradient Boosting Machine (GBM); 4) Support Vector Machine (SVM) and 5) Extreme Gradient Boosting (XGB). FIG. 11B shows ML regression results for the first- and second-best performance models: 1) Random forests (RF); 2) Extreme Gradient Boosting (XGB).

FIG. 12 shows Principal Analysis for Area Under the Receiver Operating Curve (ALTROC) characteristics of the different methods trained classifying waveforms as absence (concentration 10⁰) or presence (concentrations 10¹ to 10⁵) of bacteria. It should be noted that, although FIGS. 11A-B, 12, and 13 show data specific to the bacterial species E. coli, the device is nevertheless capable of identifying multiple separate bacterial species. This is the result of each bacterial species having a specific spectrometric signature that can be identified by the algorithms utilized for detection. In other words, the method can distinguish between different bacterial species

FIG. 13 shows results of the most accurate of the various algorithms that were tested, support vector machine (SVM) analysis, for absence results on all selected metrics (concentration 10⁰) or presence (concentrations 10¹ to 10⁵) of bacteria (E. coli in this example).

FIG. 14 shows Principal Analysis AUROC for two biomarkers in a Liquid bio sample (Nitrates (Biomarker 1) and Leukocyte esterase (LE, Biomarker 2)). The graphs show Area Under the Receiver Operating Characteristics (AUROC) of the different methods trained classifying waveforms as absence or presence of nitrates or LE in liquid bio samples, where the following acronyms apply: LR, Logistic Regression; RF, Random forests; GBM, Gradient Boosting Machine; SVM, Support Vector Machine; XGB, Extreme Gradient Boosting.

FIG. 15 shows support vector machine (SVM) analysis for two biomarkers in a Liquid bio sample (Nitrates (Biomarker 1) and Leukocyte esterase (LE, Biomarker 2)). Presented are the primary analysis performance metrics for the best performing method, Support Vector Machine. Abbreviations include: AUROC, Area Under the Receiver Operating Characteristics; Sens, Sensitivity; Spec, Specificity; F, F-Score.

FIG. 16 shows determination of flow rate which is calculated by measuring a weight change in the catheter bag during biofluid flow as a function of time.

FIG. 17 shows an example of a system for biofluid monitoring and analysis in accordance with some embodiments of the disclosed subject matter.

FIG. 18 shows an example of hardware that can be used to implement a computing device and server in accordance with some embodiments of the disclosed subject matter.

FIG. 19 shows an example of a process for biofluid monitoring in accordance with some embodiments of the disclosed subject matter.

### DETAILED DESCRIPTION

Healthcare delivery is complicated by the fact that medical treatments are often inconsistent and highly dependent on the complexity and the level of attention that is required for the delivery of care. It is somewhat surprising to find that often more complex tasks are performed with a much higher level of effectiveness than simpler ones. This is a result of the fact that complex tasks are part of what is known as a cognitive hypervigilant state whereas simple tasks are relegated to an inattentive state. This is part of the reason why healthcare performs at higher levels of delivery when dealing with complicated tasks such as transplant surgery while simultaneously delivering very poorly on the elimination of simple failures such as medication errors and the avoidance of hospital acquired infections (HAIs).

As noted above, hospital acquired infections are a major problem in modern healthcare. Prevalence of these HAIs in the United States alone is measured at 1.7 million infections per year with a human cost that translates into 99,000 deaths. The financial toll in the United States is measured somewhere near $45 billion a year. It is clear then, that these infections pose a serious human and financial cost to the American healthcare system. This of course, is not a problem restricted to the United States and is seen throughout the developed and developing world.

Catheter associated urinary tract infections (CAUTIs) represent one of the most common forms of HAI. They represent approximately 32% of all HAIs affecting over 2.5 million patients per year with a high level of morbidity and a mortality rate of approximately 13,000 deaths per year. It is estimated that each of these infections has an additional cost of $13,731 per case. In aggregate the cost to the United States healthcare system for catheter associated urinary tract infection approaches $7.7 billion per year. Given this, it is clear that the financial and human cost of this CAUTIs is significant.

The reasons why we have not been able to resolve the seemingly simple problems of HAIs and specifically that of CAUTIs are both human and technological. The human factors include several heuristics that are pervasive in medicine, the most significant being the “status quo bias”. We believe these infections to be part of the standard, recognize complications of practicing medicine—in other words, we believe that this is the “cost of doing business” and therefore, unchangeable. The technological problem is that up until now, there has been no simple, effective, real time and continuous system to monitor for potential catheter associated infections. This combination of human factors and technological deficiencies has left us in a position where nobody has truly sought a solution to this truly significant problem.

It is clear that what is required to resolve this problem is something that overcomes human and technological issues. Any possible real time monitoring solution to CAUTIs in the current state is impractical and non-implementable as it would involve too many steps, too many people, and too much time that at the end of the day would not deliver actionable data capable of preventing negative outcomes. In other words, any current solution imposes too much of a cognitive and operational load on the clinical system without providing patients or providers benefit. This truth makes the realistic implementation of a solution utilizing existing tools extremely unlikely.

Likewise, it would be impossible to create a monitoring solution for all the infections related to patient delivered, home medical care procedures that depends on active participation of patients in this process. In this setting, the manipulation of catheters and bio samples that would be required of patients would likely increase the risk of infection rather than decrease it.

It is evident that what is required to resolve this major health issue is an innovation which brings a mobile, continuous, point of care, disposable, and cost-effective solution to bear. What is called for is a non-invasive monitor which provides continuous screening and diagnosis for decision support and treatment modification. The solution described in this application makes this possible by leveraging the analysis of bacteria and biomarkers in liquid bio sample at the patient's bedside in real time. We will do this with virtually no additional clinical workload, which improves adoption of the technology, and by providing real-time, actionable data with guidelines-based decision support. Effectively, this solution will release much-needed cognitive and operational bandwidth from healthcare teams. We do this by continuously monitoring bacteria, identifying specific strains and their concentrations, identifying biomarkers associated with active infection, and translating these results into clear, data-driven decision support. In various embodiments, one or more biomarkers related to the functional status of a particular bodily system (e.g. one or more of the cardiac, respiratory, renal, neurologic, endocrine, or immune system) may be identified and the patient's status may be determined based on these biomarkers. In some embodiments, a machine learning system may be trained to identify these and other biomarkers related to the functional status of the one or more bodily systems of the patient to assist the user (e.g. clinician) with evaluation of the patient's status. The biomarkers that have been identified (e.g. using a machine learning algorithm) may assist the user with the identification of at least one condition of the patient (e.g. a condition of one or more of the bodily systems of the patient) and the patient status may be determined based on the identified at least one condition.

Accordingly, in one embodiment the invention includes a device including a spectrometer which attaches to a drainage tube of a medical tube (e.g. a urinary catheter or a peritoneal dialysis catheter) and obtains measurements (e.g. measurements of absorbance in the IR) continuously without a need to block the flow of fluid in the tube. The data from the spectrometer is analyzed in order to identify one or more materials in the patient's liquid bio sample including bacteria (e.g. E. coli), leukocyte esterase (LE), and nitrates. The data, which may include information pertaining to absorbance as a function of wavelength, can be analyzed using principal component analysis or various AI classification models.

Since the creation and modern usage of indwelling drainage catheters started in the 1930's we have seen virtually no change to the nature in management of these catheters. Therefore, to advance this technology the systems and methods disclosed herein apply new sensors based on non-invasive spectrometry techniques and combines this with artificial intelligence data analytics to provide a breakthrough development for continuous infection surveillance. This ability to detect the existence of bacteria in any biofluid sample such as liquid bio sample without interacting or directly manipulating the sample itself has tremendous value in modern healthcare. Such a capacity will allow for continuous sampling of specimens without altering or adding to the workflow of the clinicians currently caring for patients. Given this passive sampling method's incorporation of continuous sampling into the workflow, we can guarantee that patients will receive continuous screening for early infection in any indwelling catheter, such as a urinary catheter. This ability to detect bacterial colonization and early bacterial infection will profoundly affect the delivery of safe care as it will eliminate many infections that are currently only identified after the existence of advanced infections.

Disclosed herein are embodiments of a continuous, real-time, on-catheter, on-patient device which can be used in a clinical setting. Embodiments of the disclosed on-catheter design include capability to communicate and interact directly with clinicians and care givers.

The following workflow and hardware elements may be used to carry out various embodiments of a continuous, on-catheter screening of biofluid for the presence of one or more known bioproducts and microorganisms:

System Workflow and Implementation:

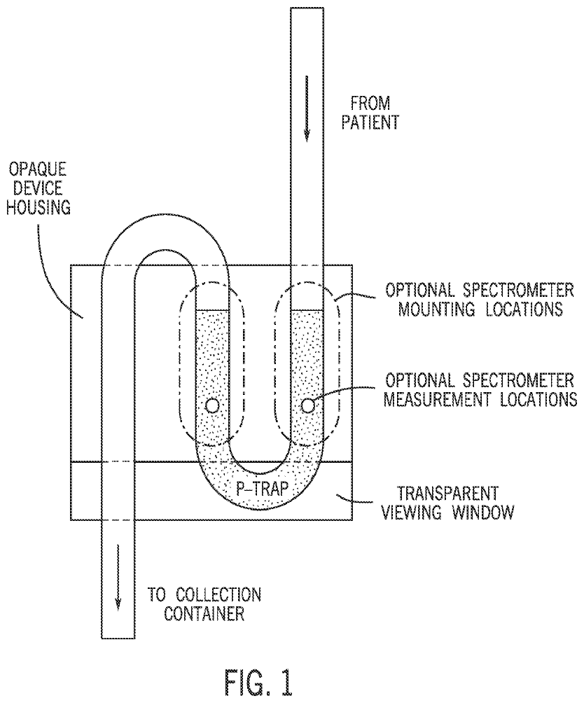

In various embodiments, the system includes a spectrometry device which can be placed on the drainage tubing of any existing or newly placed indwelling patient catheter with external drainage. This is accomplished by including the following elements (see FIGS. 1-4):
a. Use of a standard drainage catheter tubing made of any common material used for such drainage catheters including rubber, silicone, latex, Polyvinyl Chloride (PVC). This is the case because the infrared spectrum in which the micro-spectrometer functions is not affected by the properties of these materials.
b. The micro-spectrometer inside of its housing/mounting hardware is clamped to the outside of the drainage catheter tubing as seen in FIGS. 1-3.
c. This is accomplished in a way that creates curvatures in the catheter that creates two impediments or blocks to the free flow of liquid bio sample—the impediments or blocks are located at each of the angulations created at the point of curvature
d. Given these impediments or blocks, there is created an area of stagnant liquid bio sample column in which the entire tubing is filled with liquid bio sample with no air; fluid can still move through the tubing but the impediments or blocks cause sufficient fluid to accumulate in one location to permit optical measurements to be made.
e. The mounting is constructed in such a way so that the micro-spectrometer sampling window is directed precisely toward this created area of liquid bio sample stagnation.
f. The bio sample can then be analyzed by illuminating the sample with the micro-spectrometer. This analysis can be performed at any frequency that is deemed clinically relevant. The time required for the micro-spectrometer to obtain data from the liquid bio-sample is less than 1 second.

No alteration to the existing catheter system is required. In particular, and of critical importance, no penetration or violation of the existing catheter system is performed as it is not required that the biofluid be in contact with any element of the device testing system. The spectrometric system uses its light characteristics to penetrate the tubing of the existing drainage system to continuously analyze the biofluid included in the tubing.

On-Catheter Device

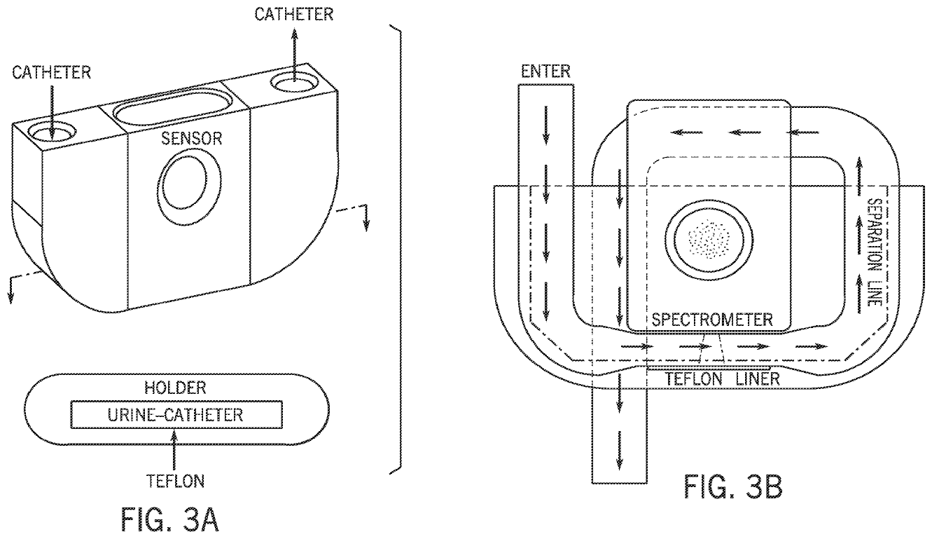

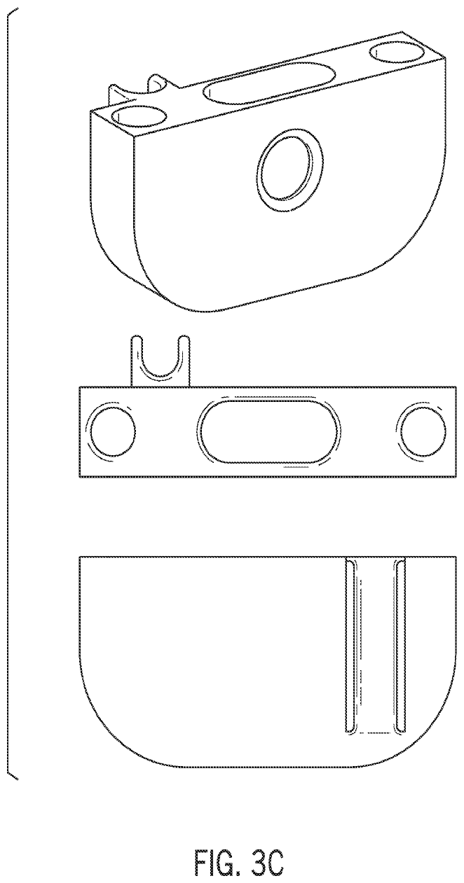

As seen in FIGS. 1 and 3, the on-catheter device may be enclosed in a housing that is clamped onto the outside of the drainage catheter system in such a fashion that it guarantees free flow of biofluids through the system while allowing accomplishment of continuous spectroscopic analysis of samples within the catheter system tubing.

In various embodiments (in which the analysis may be accomplished without using blocks to the flow of biospecimen for the purposes of creating a temporary non-flowing biospecimen sample used for obtaining a spectrometric data set), the analysis may be accomplished by using the natural bending properties of the drainage catheter tubing to collect a sample of fluid by gravity, for example at a low point in the tubing. Fluid from the urinary catheter may move through the tubing in drips or a small trickle, which in a vertical segment of tubing would move past the IR absorbance sensor too quickly to obtain a stable reading. Therefore, creating a low point (e.g. a horizontal portion or a bend) in the tubing this ensures that a small amount of fluid will be retained for a sufficiently long period of time (e.g. for at least several seconds or tens of seconds) to allow absorbance readings to be taken.

The tubing material naturally forms non-occlusive bends through which the liquid bio sample regularly flows through vertical segments and stagnates in curved or horizontal segments. Using this property, the mounting apparatus demonstrated in FIGS. 1 and 3 will create a gentle curvature in the catheter tubing so that an area of biospecimen stagnation, or accumulation, may be created. This will allow sampling of the biospecimen with the spectrometer for the period of testing which is on the time order of 1 second for testing of a sample. In this embodiment, the spectrometer will be positioned in the center of the bend formed by the mounting device, with the sampling window facing the area of stagnant bio sample created by the mounting device (e.g. in the area of the tubing labeled “p-trap” in FIG. 1). By creating an area (e.g. a bend) within the tubing in which fluid is still flowing but is sufficiently slowed or stagnant to permit spectroscopic measurements, this helps ensure that a data set of a fresh bio sample will always be available for testing with the device, since new fluid material will continue to enter the measurement area. In contrast to certain prior systems which employ one or more blocks to accumulate sufficient fluid to obtain optical readings of the sample, the present invention uses a bend such as a p-trap to collect fluid for analysis without having to block flow, which simplifies the design of the device and facilitates continuous measurement of the patient's sample. A spectrometer (which may include a light source and detector, as described in more detail below) may be mounted in the housing in any location at which fluid will accumulate. Accordingly to various embodiments, two possible spectrometer mounting locations are indicated in FIG. 1 by the vertical ovals, with circles showing the possible locations at which spectrometer measurements may be obtained. In certain embodiments, the housing may be opaque, which facilitates spectrometer measurements by reducing potential background light contamination. A transparent viewing window may be located near the bottom of the housing to facilitate insertion of the tubing as well as to allow a user to confirm that fluid is moving through the tubing associated with the housing. In some embodiments, the bend in the tubing at which fluid accumulates may be referred to as a p-trap.

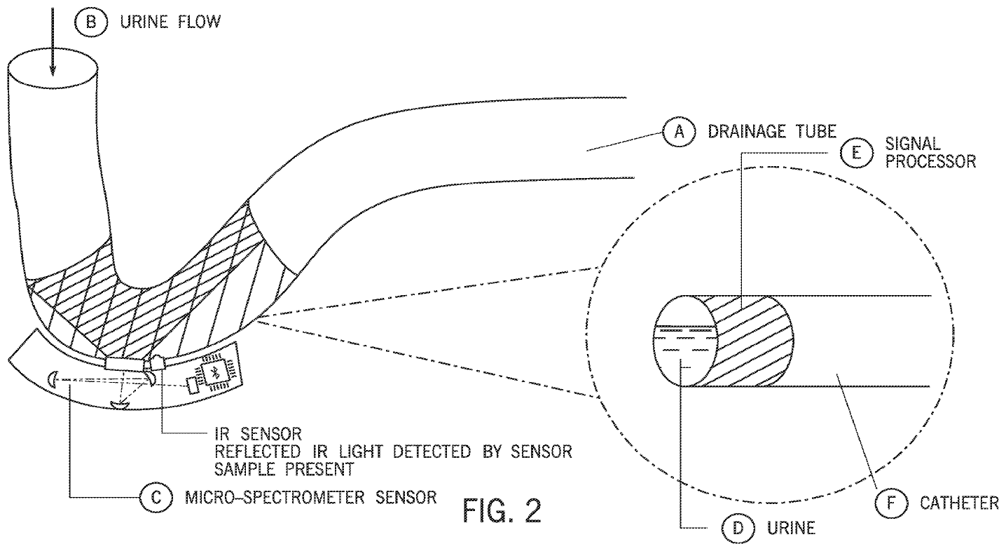

FIGS. 2 and 3 provide a close up view showing how the sensor will interact with the drainage catheter tubing in certain embodiments. The embodiment of FIG. 2 in particular provides labels showing how the interacting with the drainage catheter tubing (A) and the area of the bio sample (e.g. liquid bio sample, (D)) accumulated in the area adjacent to the sensor. The left portion of FIG. 2 shows a segment of catheter tubing (F) into which liquid bio sample is flowing (B) while the inset on the right shows a liquid bio sample (D) accumulated inside the tubing as well as a signal processor (E). In this embodiment, the housing includes a curved face which is located adjacent to the bend in the tubing such that the tubing and the housing are closely aligned, which optimizes the transmission of light from the light source to the sample within the tubing as well as the transmission of light returning from the sample to the sensor or detector in the housing. The segment of tubing includes a bend or p-trap, situated so as to create a low point in the flow path, and is shown as having an accumulation of fluid (liquid bio sample, (D)).

On the outside of the tubing below the p-trap, a micro-spectrometer sensor (element C in FIG. 2) is attached adjacent to the tubing in a location adjacent to the low point where fluid accumulates. Infrared (IR) light is emitted from the sensor device into the tubing and an IR sensor adjacent to the tubing measures IR light reflected by the sample inside the tubing. In addition, the micro-spectrometer sensor (C) measures spectra (e.g. absorbance spectra) in the IR range from the sample. The frequency interval of data collection and what will be considered to be “continuous” sampling will be determined by clinical needs for a particular situation and patient, and the system will be capable of adjusting to these requirements. In various embodiments, continuous sampling may include sampling at least once per minute, once per 30 seconds, once per 10 seconds, once per second, five times per second, or other more or less frequent sampling intervals as called for in the particular situation.

FIGS. 3A-3C disclose an embodiment of an on-catheter sensor system design. FIG. 3A shows a perspective view (top) of the sensor housing and a cross-sectional view (through line A-A′ in the top view) showing an accumulation of liquid bio sample (e.g. urine or peritoneal fluid) in the catheter tubing in the region below the sensor. The housing in FIG. 3A includes a central oval-shaped opening in which a sensor device may be inserted. FIG. 3B shows a side view of the sensor housing with a sensor device inserted from the top into the oval-shaped opening. The housing also includes openings on the sides through which catheter tubing may be inserted, where the inserted tubing traces an approximately U-shaped path through the housing, entering on one side and exiting on the other side, to provide a low point at which liquid bio sample accumulates and can be monitored. The side view of FIG. 3B shows the housing and sensor with a section of catheter tubing running through the housing. The tubing enters the housing from the top left, travels through the housing in a U-shaped path, and exits the housing from the top right. Upon exiting the housing, the tubing may then complete a loop and attach to a clip on the side of the housing (see FIG. 3C) to stabilize the tubing. The housing may include a liner (e.g. made from Teflon) to block light from exiting the housing in order to minimize or prevent contamination of the light signals originating from the liquid bio sample with spurious signals which might arise from nearby materials outside the tubing. In various embodiments, this design may include a slot into which the catheter tubing may be inserted such that a portion of the catheter tubing is adjacent to the spectrometer (see FIG. 3B).

The housing may include a window on the side which aligns with an indicator on the inserted sensor device. The window on the housing may be just an opening or may include a lens that may be flat or curved to permit light signals from the sensor device to be seen, where the curved lens allows the light signals from the sensor device to be seen at a wider range of angles. In some embodiments, a portion of the side of the housing may be removable (e.g. along the dotted “separation line” shown in FIG. 3B) to allow the catheter tubing to be inserted into the U-shaped track from the side. The side portion may then be reattached to help keep the tubing in place and also to maintain a low-light or light-free background in the vicinity of the spectrometer sensor. Permitting the tubing to be attached from the side in this way allows the sensor housing to be attached to a catheter tubing that is currently coupled to a patient in a way that does not require decoupling the tubing or interrupting drainage.

FIG. 3C shows a perspective view (upper panel), a top view (center panel), and a side view (bottom panel) of the housing. These views depict the clip (on the left in the upper and center panels, on the right in the bottom panel) to which the tubing may be attached to provide additional stability.

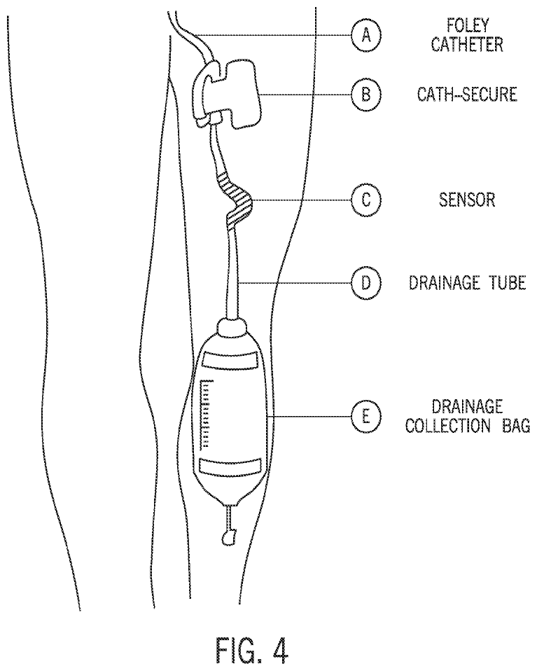

FIG. 4 provides a diagram of a system such as that of the embodiment of FIG. 2 in the case that the bio sample being utilized is a liquid bio sample coming from the patient's bladder or peritoneal cavity. The diagram of FIG. 4 shows a Foley catheter (A), a catheter securing device (B), a sensor (C) (e.g. shown schematically as a U-shaped bend but which may be mounted in a housing such as that shown in FIG. 3) located at a bend in the drainage tubing (D), and a drainage collection bag (E). In the case of other bio samples besides urine, the arrangement would be similar to that shown in FIG. 4 but would be suitably adapted based on the site of origin of the bio sample. Other potential bio sample sources include peritoneal fluid from patients undergoing peritoneal dialysis, biliary fluid from patients undergoing biliary system diversion, naso/oral enteric tubes in patients undergoing enteral decompression, and surgical drainage in patients with surgical wounds drains.

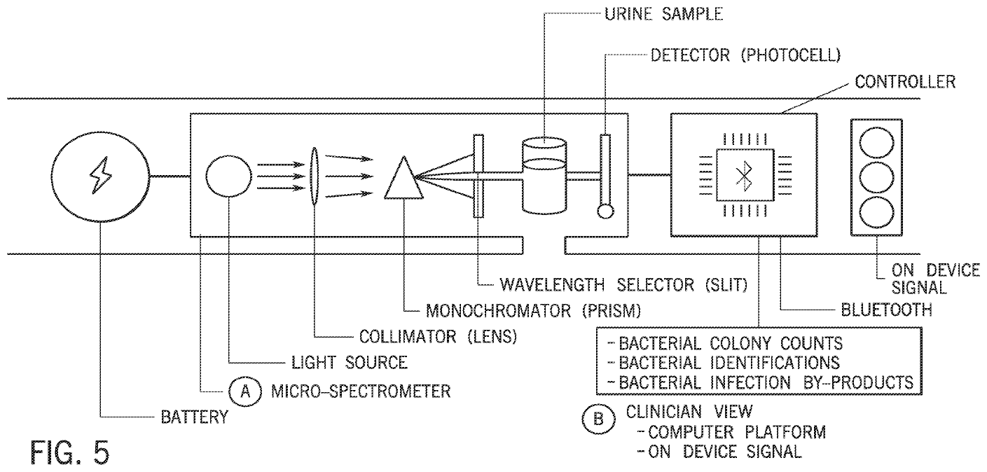

FIG. 5 provides a detailed view of an embodiment of the on-catheter device. The device includes a control system arranged and adapted to carry out the procedures disclosed herein and which includes a catheter holder for the device. Also included is a power source (e.g. battery power for mobile deployment) and in one particular embodiment the device may include a lithium ion battery that will be rechargeable and able to hold a charge for continuous (24/7) usage for up to 14 days. In another embodiment, the lithium ion battery may not be rechargeable but nevertheless will be able to hold a charge for 24/7 usage for up to 14 days.

The device also includes a micro-spectrometer to generate data which can then be subject to further analysis on or off the device (or both). The spectrometer may include one or more light sources such as light-emitting diodes (LEDs) to emit light into the sample in order to obtain data. In one embodiment, the spectrometer may use different LEDs to select for the ideal waveform for the identification of specific bacterial strains as well as separate biomarkers. The number of LEDs can vary depending on the products (e.g. bacteria and biomarkers) one wishes to identify.

In various embodiments the spectrometer may include, along with the light source to illuminate the sample, a collimator (e.g. lens) to concentrate the light within the sample. The spectrometer may also include a monochromator (e.g. a prism), to divide the light sample into its constituent wavelengths, and a wavelength selector (e.g. a slit), to select the correct wavelength for selected bacterial strains or other products of interest.

In general, the patient's liquid bio sample (e.g. urine, peritoneal fluid, wound drainage, enteral content, etc.) will remain at all times under the following conditions: inside of the drainage catheter tube and completely separate from the device with no element of the device coming into contact with the bio sample.

The spectrometer also includes a detector (e.g. a photocell) which records the wavelength results of light returning from the sample from each illumination (e.g. absorbance). The spectrometer may also include a communication module (e.g. a Bluetooth device) for optional transmission of data and other information to remote device such as a computing platform, which can include an electronic health record and/or a stationary or mobile computing device where clinicians can see results and updates on the patient's status. This digital result can be shared with any number of clinicians and administrators who have been cleared though concerns relating to patient privacy to manage the patient's clinical status as well as to manage broader infection control issues related to the institutional concerns.

The procedures for sharing these digital records will generally be determined by the managing team caring for the patients but can include, but are not limited to, the use of messages posted in the medical record, text messages, pages, and phone calls to responding clinicians and administrators, among various possible means.

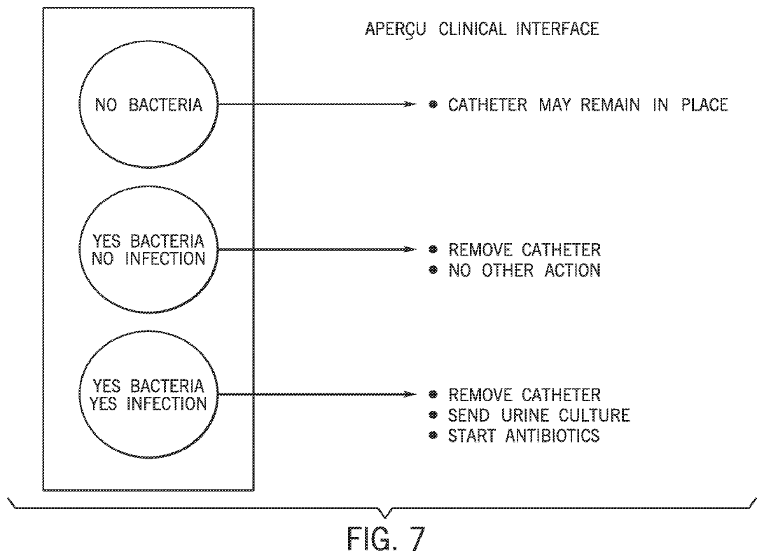

The device may also include an on-device signal system so that users such as clinicians can visualize results without leaving the patient's setting. The signaling system may be located at or visible from the patient's bedside and may include a status of the patient's bio sample as well as a recommended management paradigm determined by the local treating team. A sample of this management could include the protocol seen in FIGS. 5 and 7, which show a simple stoplight type signal system which may include one or more status indicators. In the examples shown in FIGS. 5 and 7, the on-device signal may include three indicator lights which may be used to indicate that the patient's status is good (e.g. a green light), questionable (e.g. a yellow light), or needing attention (e.g. a red light); these conditions may be related to State 1, State 2, and State 3 described below for FIG. 7.

Data from the on-device indicator may then be transmitted (alone or along with other information) to a remote computing platform (e.g. a mobile or cloud-based computing platform) to perform further analysis such as spectral analysis and which in some embodiments may be analyzed using machine learning algorithms to detect the components of the spectra that are emitted and captured.

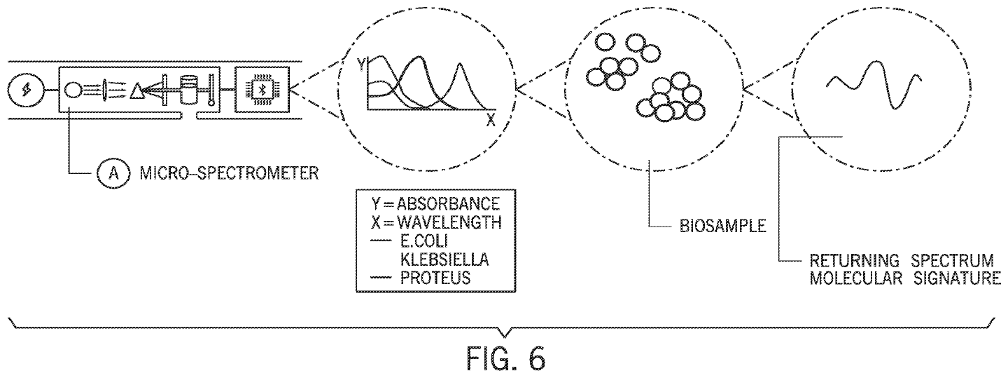

The remote computing platform may process the data using machine learning algorithms to provide results regarding, bacterial colony count, bacterial colony type, and bacterial infection by-products identifiable in the sample, among other information. As seen in FIG. 6, each bacterial species (and even the concentration of each bacterial species) and biomarker may have a different spectral signature related to the compound and the wavelength of light utilized by the spectrometer. For example, the diagram in FIG. 6 depicts spectral data being transmitted from the spectrometer corresponding to E. coli, Klebsiella, and Proteus bacteria.

In various embodiments, the combination of the results of analysis can yield three clinical entities (see FIG. 7):
State 1: No bacteria or infection in bio-sample
State 2: Bacterial colonization but no infection in bio-sample
State 3: Bacteria and infection in bio-sample

Clinicians according to their experience and particular practice patterns will determine their clinical response to these distinct states.

ML Algorithms

Machine Learning (ML) algorithms are developed to be used specifically with the device disclosed herein rather than independently of the device. The algorithms will be utilized to perform the analysis of the sample's waveforms and are constructed as follows:

Every sample contains a waveform made of 330 data points. The device performing data analytics using machine learning comprises:
One module for classification
One module for sensitivity analyses
An unsupervised learning module configured to generate the final result based on the organized data set

The classification module may perform one or more of the following: extract data, which may be performed continuously from the patient's bedside using the spectrometer hardware; load the extracted data into a dataset; and generate results based on the colony forming units (CFUs) in the sample.

The extracted data may be classified using one or more of the following classification methods: Gradient Boosting Machines, Support Vector Machines, Random Forests, extreme Gradient Boosting, Logistic Regression; and Random Hyperparameter Tuning in 10 folds cross-validation (CV).

The ML algorithm creates two or three groups based on the CFUs and assigns the samples to each of the respective groups. The output of the ML algorithm includes bacterial concentrations expressed in a range from 10⁰ to 10⁵, where 10⁰ means the absence of bacteria, and from 10¹ to 10⁵ indicates the amount of concentration of the present bacteria within the sample. Predictive performance of the ML algorithm may be assessed by determining AUROC, Precision (AP), specificity, sensitivity, and F for each one of them.

The regression includes a quantification of bacteria metrics using different datasets in which 10⁰, 10¹, 10², 10³, 10⁴ and 10⁵ are marked as 0, 1, 2, 3, 4, and 5 respectively, creating a continuous outcome in which, by using such waveforms, any given concentration is predicted (0-5). Regression models addressed included Random Forests, Extreme Gradient Boosting, Linear Regression, Elasticnet, Lasso and Elastic-Net Regularized Generalized Linear Models. A Random Hyperparameter Tuning in 10-fold cross-validation is also performed for a greater R-squared.

Sensitivity Analysis Module

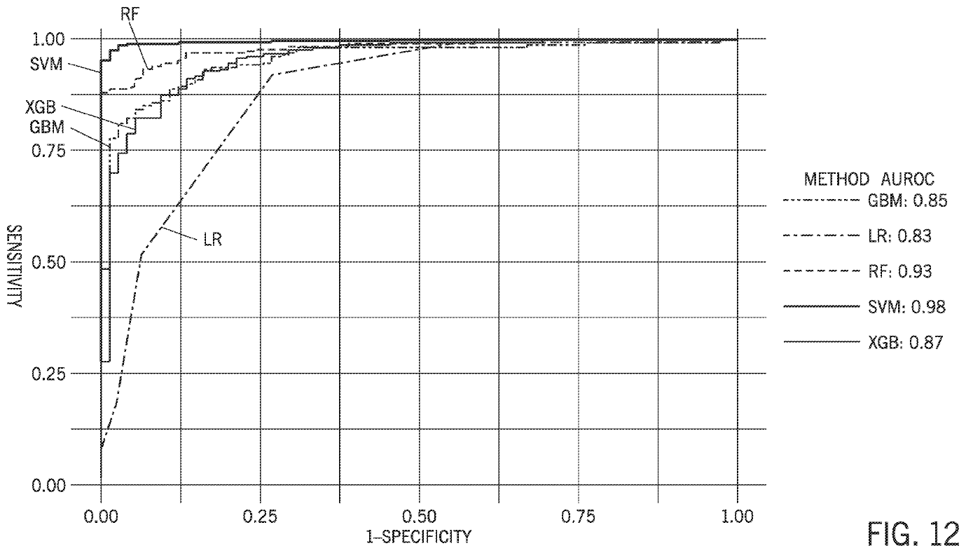

The Sensitivity Analysis Module may be used to determine the presence or absence of bacteria in a fluid based on a continuous steam of data from the device, data regarding possible outcome of the bacteria concentration. To test the sensitivity of the device, various tests were performed to compare different concentrations of bacteria. Eight databases were created based on these numbers. In dataset number one, 10⁰ was considered to be an absence of bacteria and 10¹, 10², 10³, 10⁴, and 10⁵ were considered as presence of bacteria in various concentrations. In dataset number two, the two groups were split as 10⁰ vs. 10¹, 10², and 10³. In dataset number three, the two groups were split as 10⁰ vs. 10¹, 10², 10³, and 10⁴. In dataset number four, the two groups were split as 10⁰ vs. 10¹. In dataset number five, the two groups were split as 10⁰ vs. 10². In dataset number six, the two groups were split as 10⁰ vs. 10³. In the dataset number seven, the two groups were split as 10⁰ vs. 10⁴. And in dataset number eight, the two groups were split as 10⁰ vs. 10⁵ (FIG. 12).

Unsupervised Learning Module

Provided below is a list of the performance metrics of all the sensitivity analyses performed for each dataset obtained. The models were trained using random hyperparameter tuning 10 folds CV and validated in the testing split containing 25% of their observations for the classification models. For the regression model, 100% of the dataset number one observations were used to perform 10 folds CV.

The primary analysis had outstanding performance achieved using an SVM. It is configured to assemble the unstructured data set into multiple versions of the organized data set. The module is configured to create training data from the organized data set and wherein the supervised learning module is configured to use the training data to generate one or more groups.

### Example

The following provides details of a non-limiting example according to embodiments of the invention, including methods and results of building and using the device to collect data and processing the data using a Machine Learning algorithm.

Under Partners HealthCare Institutional Review Board approval, two hundred samples were analyzed from September 2018 to January 2019 at Harvard Medical School Microbiology Laboratories and Brigham and Women's Hospital. Statistical analyses were performed in R version 4.0.0 and RStudio version 1.2.5019.

Bacteria Analysis

Serially-diluted samples were prepared using a culture of Escherichia coli MG1655 and synthetic urine (Pickering laboratories 1700-0600). Twenty-four hours before the experiment, 5 mL of EZRDM media (Teknova) were inoculated with 10 uL of a saturated E. coli culture and incubated overnight at 37 C with 220 Revolutions Per Minute (RPM). Dilution series were created by diluting 500 uL of the culture medium in 4.5 mL of synthetic urine, vortexed for 5 seconds, and then 500 uL were transferred into 4.5 mL of synthetic urine. Each subsequent dilution was created utilizing the same protocol until a total of 10 dilutions were reached. One sample in each group was left without any bacterial inoculation as a control. Spectrometry samples were prepared by transferring 4 mL of each dilution to glass spectrometer cuvettes sealed with Parafilm. Determination of the concentration of bacteria in the prepared synthetic urine samples was accomplished by plating 100 uL of each sample in LB agar to determine colony forming units (CFUs). Each plate was incubated overnight at 37 C, and colonies were counted the following morning using a proprietary machine learning algorithm to automatize and standardize this process. Both the spectrometry and the microscopic readings were performed simultaneously to avoid any discordance in the time from sample preparation to sample analysis.

3-D Printing

The integrated spectrometer and liquid bio sample holder (see FIG. 3) were designed in Solidworks (Dassault Systemes, Velizy-Villacoublay, France) and produced using an Objet30 3D printer (Stratasys, Eden Prairie, Minnesota, USA) from a white photopolymer resin (RGD835). The design was created to simulate a mount that could be attached to the outer surface of a urinary drainage catheter. In order to reduce background signal contamination, the cuvette holder had an integrated (opaque) lid and a 3 mm-thick Teflon liner that blocked the light path from exiting the cuvette/catheter.

All of the samples, each having a different concentration, were analyzed using the device described in this application and, in parallel, simultaneously underwent microscopic colony count analysis to represent the gold standard for bacterial colony count identification.

The device was used to obtain spectrometric data, perform chemometric analysis, and create calibration models for bacterial detection. The data from both methods was recorded in separate databases and correlated with appropriate sample identifiers. Raw data from the spectroscopic evaluation was analyzed and incorporated into the Machine learning algorithms, using the microbiological colony counts as the representation of the gold standard accurate results.

Data Analysis

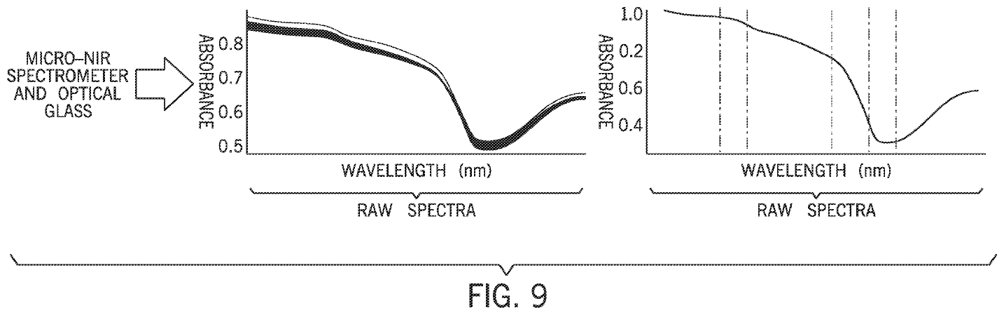

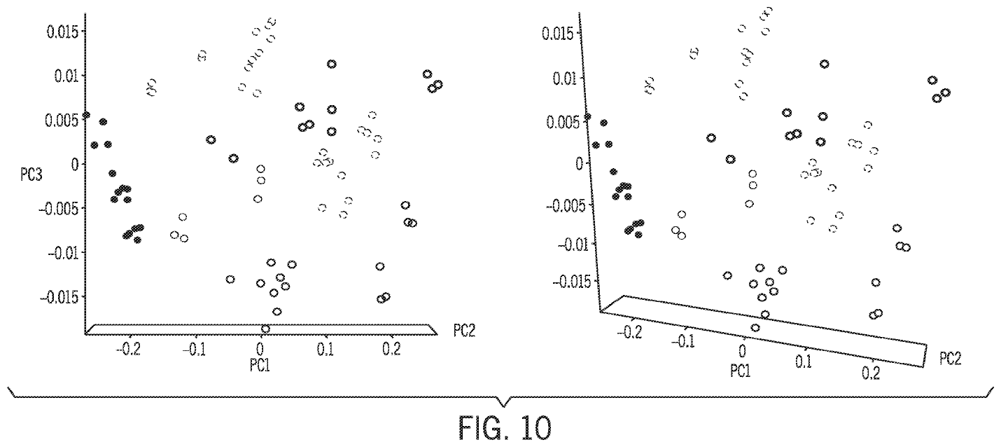

Bacteria concentration ranged from 10⁰ to 10⁵, where the exponent indicates the number of bacterial CFU in the sample. Every bacterial colony concentration has a characteristic morphologic waveform signature determined by the combination of CFU and the wavelength utilized to analyze the sample. This signature waveform is created from 330 separate data points (see FIG. 9, with wavelength in nm on the x-axis and absorbance on the y-axis) and from principal components analysis (FIG. 10). FIG. 9 shows the results of the data processing from raw to processed data. In this process we selected the wavelength from 740 to 1070 nm, then processed and normalized the data.

Processing: Assumes Beer-Lambert model is valid and transforms the measured signal to be linear with concentration by doing a log transform and adjusting the result for noise and deviations from the model.

$$A = {log}_{10} \frac{{I}_{o}}{I}$$

FIG. 10 depicts results from a Principal component analysis, or PCA, which is a dimensionality-reduction method that is often used to reduce the dimensionality of large data sets, by transforming a large set of variables into a smaller one that still contains most of the information in the large set. In our setting the PCA approach has taken information related to different concentrations of bacteria in a liquid bio sample and classified them based on the number of CFUs. It was used it as an exploratory tool for our analysis as it demonstrates the grouping of different CFU concentrations into well-defined clusters.

In addition to bacterial detection, it was felt that the addition of detection of biomarkers associated with urinary tract infection (UTI) would add to the value of the prediction models. This would allow for the establishment of three separate clinical states: 1) A catheter with no bacteria and no infection, 2) a catheter with bacterial colonization and no evidence of infection and 3) a catheter with bacterial infection. Based on this concept, two sensitivity analyses using urinary nitrates and leukocyte esterase (LE) were performed to determine how these target variables can affect the signature waveform for each concentration.

Predictive models were created using machine learning algorithms in order to identify the smallest absolute amount of change in bacteria concentration that can be detected by our spectrometer. To increase accuracy and precision within our models, all the data used for the classification algorithms were sampled randomly and had a distribution of 75% of the data designated for training and 25% for the testing of the algorithm.

Model Training and Validation

Based on our outcome of interest, we divided the analysis of the samples in two groups with ten different models (classification models and regression models). Classification models were used to predict the concentration among different concentrations, whereas the regression models were used to predict the specific concentration of bacteria derived from their waveforms.

All models were trained using a seed so that the predictions could be replicated. We performed Random Hyperparameter Tuning in 10-folds cross-validation (CV) aiming for the highest Area under the Receiver Operating Characteristic Curve (AUROC) when training classification models and aiming for the highest R² when addressing the regression models.

Classification Models

We trained five different models in this category: 1) Logistic Regression; 2) Random forests (RF); 3) Gradient Boosting Machine (GBM); 4) Support Vector Machine (SVM), and 5) Extreme Gradient Boosting (XGB). The models used 75% of each dataset for training purposes and 25% for validation to address the most optimally trained classification model's performance.

Regression Models

We used five different models that included 1) Random Forests; 2) Extreme Gradient Boosting; 3) Linear Regression; 4) Elastic Net; and 5) Lasso and Elastic-Net. These models were trained using 100% of the observations in order to predict the different bacteria concentration levels (from 10⁰ to 10⁵), and biomarkers using the different waveforms.

Results

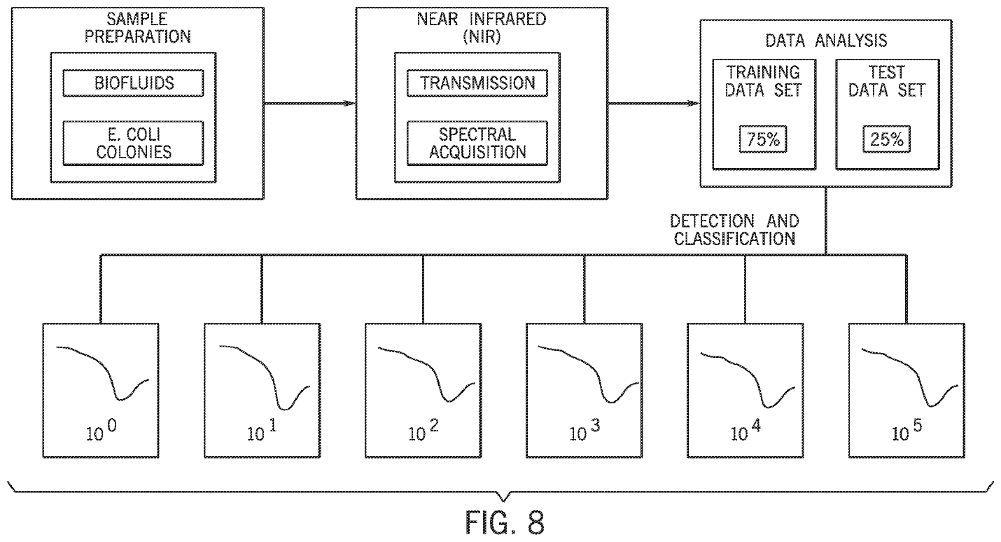

FIGS. 8 and 9 show the workflow (FIG. 8) and raw and pretreated spectra (FIG. 9) of the bacteria concentration in urine samples using a micro-near infrared spectrometer. The NIR-spectrometer scanned through a glass cuvette. The wavelength range of 740-1100 nm was found to contain the most important peaks in the spectra based on the literature.

A combination of synthetic urine and five different bacterial concentrations was analyzed for a total of two-hundred samples in the main analysis, and with four-hundred samples for biomarker analysis (200 samples with nitrates and 200 leukocyte esterase).

Principal Analysis—Bacteria Only

To validate our hypothesis, a series of experiments were conducted to observe how the CFUs of E. coli affected the waveform data in each concentration. Ten machine learning models were used to classify and established a cut-off point between samples.

Classification—Bacteria Only

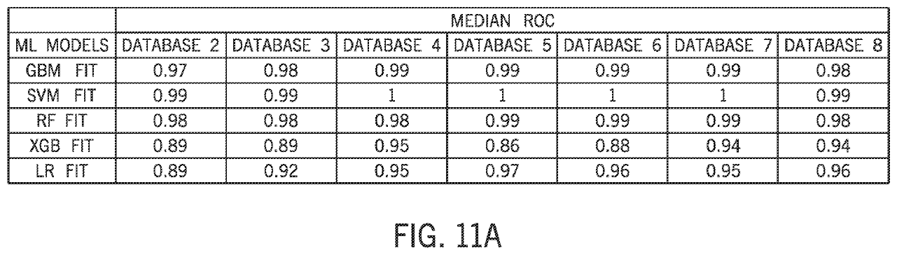

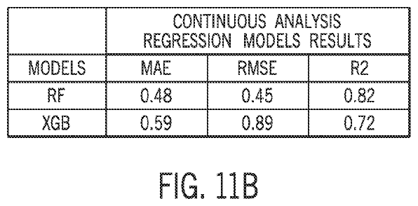

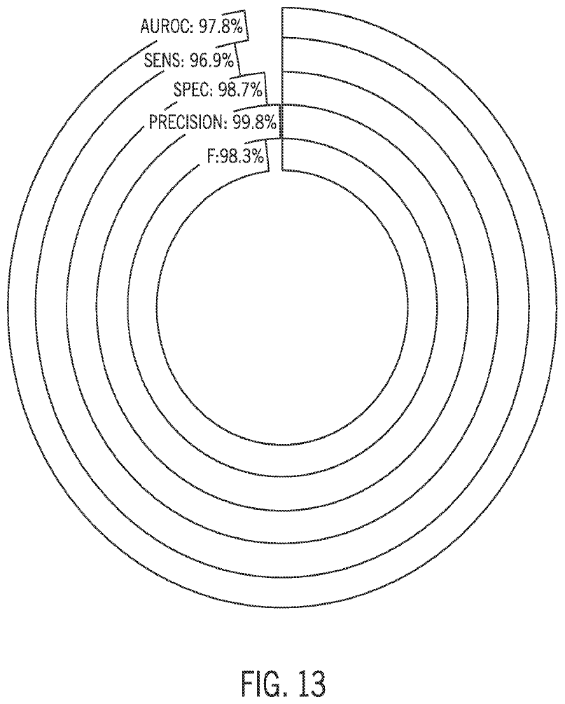

Among the five classification methods, Support Vector Machine (SVM) achieved the highest performance with a specificity of 0.99, sensitivity of 1, precision of 0.99, F-score of 0.99 and AUROC of 1. Metrics of the thirty-five different classification models assessed as part of the classification sensitivity analysis are reported in FIGS. 11A and 11B. Principal Analysis for Area Under the Receiver Operating Curve (AUROC) characteristics of the different methods trained classifying waveforms as absence (concentration 10°) or presence (concentrations 10¹ to 10⁵) of bacteria are shown in FIG. 12. The results on all the metrics selected for the most accurate algorithm are shown in FIG. 13.

Regression—Bacteria Only

The prediction performance of the regression models was addressed using R², Root Mean Squared Error (RMSE), and Mean Absolute Error (MAE).

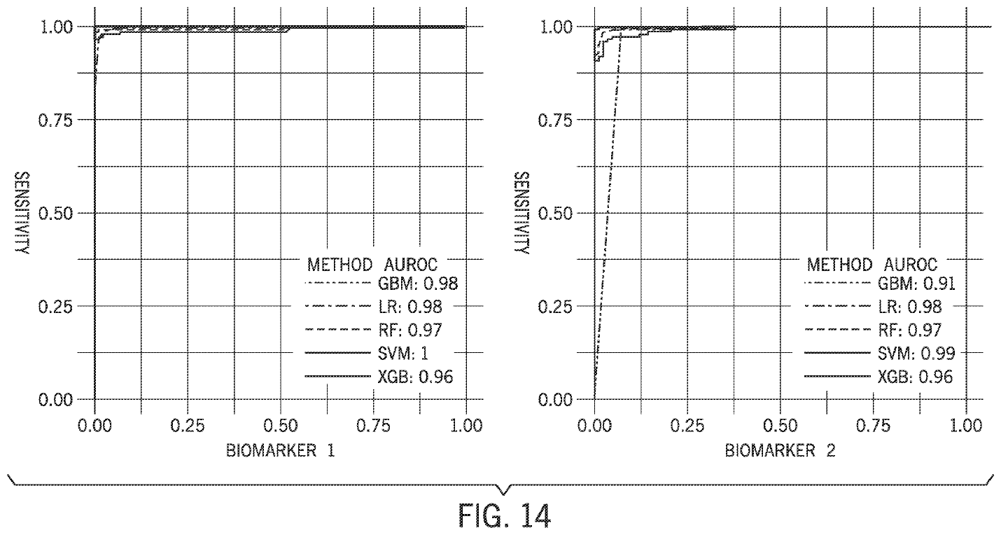

Of the five different models tested using the waveform data by means of Cross validation for regressing the bacteria concentration, the best performing one was obtained using a Random Forests method; with a MAE of 0.48, RMSE of 0.45, and an R² of 0.82. Metrics of the five different regression models assessed are reported in FIG. 14.

Sensitivity Analysis—Biomarkers

Performance metrics of the different models trained as part of the sensitivity analysis using three concentrations of nitrates and one of leukocyte esterase were analyzed as part of the sensitivity analyses.

Classification—Biomarkers

Biomarkers were classified in this work. We selected two biomarkers of many available because of their wide acceptance in clinical practice and broad adoption and availability. The two biomarkers chosen were Nitrates and Leukocyte esterase (LE), whose use in the diagnosis of urinary tract infections is universally accepted. Nevertheless, in various embodiments any Biomarker can be characterized through use of this process.

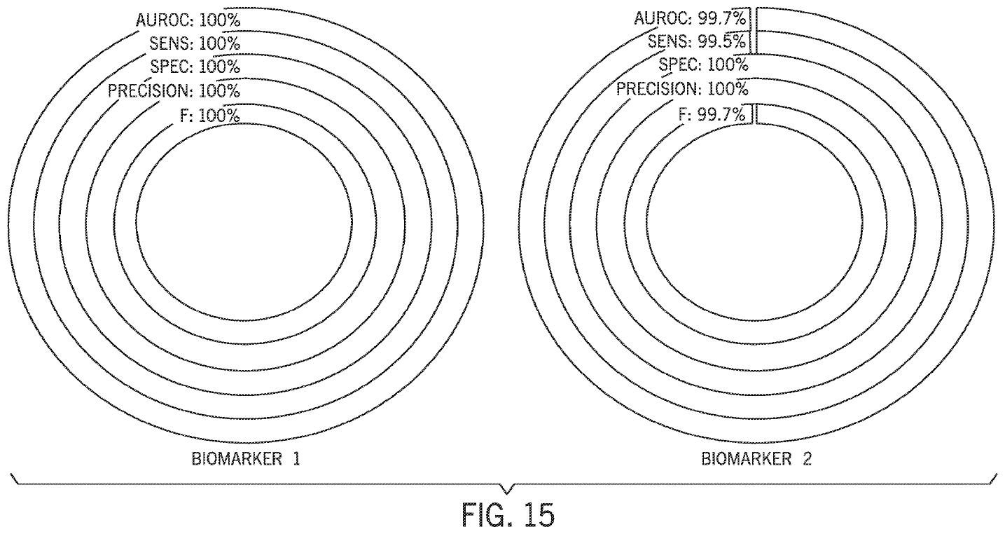

Nitrates were classified into two groups, presence or absence in urine, and were evaluated in 200 samples. All the data obtained sensitivity and specificity close to 100% in each test and high AUROC with SVM, GBM, LR mainly (see FIGS. 14 and 15).

On the other hand, Leukocyte esterase was measured with three different concentrations (1 ml of 0.45 mg/l liter of saline solution plus 3 ml of urine, 2 ml of 0.45 mg/l liter of saline solution plus 2 ml of urine, and 3 ml 0.45 mg/l liter of saline solution plus 1 ml of urine) in 200 samples in total. With our other sensitivity analysis, the support vector machine was the best algorithm with an AUROC of 0.99, followed by LR with 0.98, the precision of 1, F-score of 0.99, and AUROC of 0.99. Sensitivity and specificity were 0.99 in all the samples analyzed (see FIGS. 14 and 15).

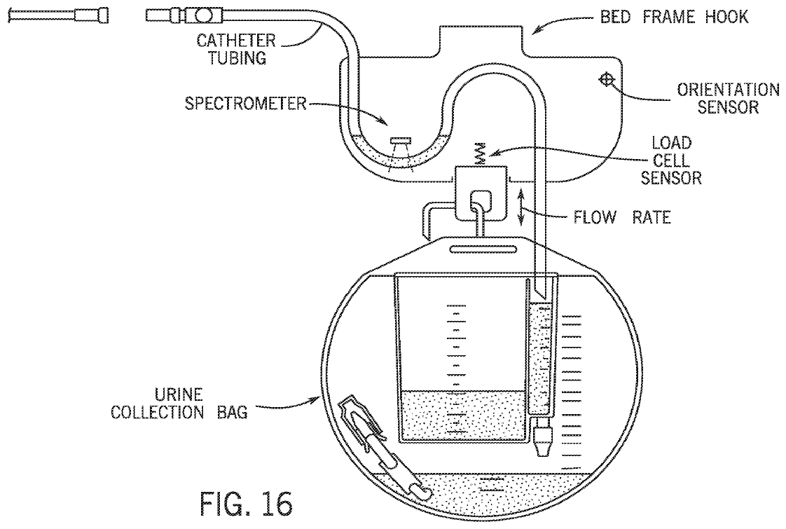

In certain embodiments, a flow rate of the biofluid may be determined using the disclosed apparatus. In such embodiments, the housing of the on-catheter sensor system (e.g. as in FIGS. 3A-3C) may include a load cell sensor or other mechanism (FIG. 16) to monitor the weight and/or changes in weight of the fluid collection bag (labeled as a urine collection bag in FIG. 16, although other fluids may be monitored using the device) attached to the load cell sensor. In various embodiments, the device may calculate an approximation of flow rate of a biofluid that passes through the indwelling catheters and therefore calculate an actual measurement of amount of biofluid coming from the patient at any given time. In certain embodiments, flow rate may be calculated through use of a measurement of changing weight in a biofluid repository (e.g. a biofluid collection bag) over time. In particular embodiments, flow rate may be calculated on a continuous basis as a measure of weight change and may be reported to the user one or more communication mechanisms of the device. In various embodiments, the calculated flow rate may be based on the following formula: δ weight/δ time. In some embodiments, an algorithm may utilize this data to calculate a volume over time calculation to yield an approximation of flow rate over time. This data may be reported to the user continuously via one or more communications mechanisms of the device. Information about the biofluid flow rate and total accumulation of biofluid may be used to monitor the patient's status.

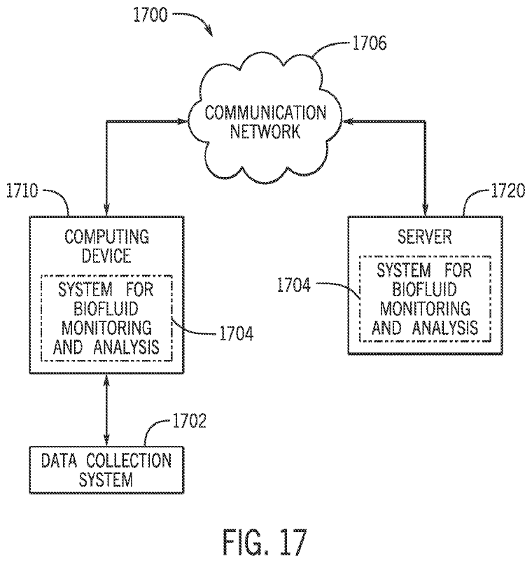

Turning to FIG. 17, an example 1700 of a system (e.g. a data collection and processing system) for biofluid monitoring and analysis is shown in accordance with some embodiments of the disclosed subject matter. In some embodiments, a computing device 1710 can execute at least a portion of a system for biofluid monitoring and analysis 1704 and provide control signals to one or more components of a data collection system 1702, for example a spectrometer coupled to a liquid bio sample system. Additionally or alternatively, in some embodiments, computing device 1710 can communicate information regarding the control signals to or from a server 1720 over a communication network 1706, which can execute at least a portion of system for biofluid monitoring and analysis 1704. In some such embodiments, server 1720 can return information to computing device 1710 (and/or any other suitable computing device) relating to the control signals for system for biofluid monitoring and analysis 1704. This information may be transmitted and/or presented to a user (e.g. a researcher, an operator, a clinician, etc.) and/or may be stored (e.g. as part of a research database or a medical record associated with a subject).

In some embodiments, computing device 1710 and/or server 1720 can be any suitable computing device or combination of devices, such as a desktop computer, a laptop computer, a smartphone, a tablet computer, a wearable computer, a server computer, a virtual machine being executed by a physical computing device, etc. As described herein, system for biofluid monitoring and analysis 1704 can present information about the control signals to a user (e.g., researcher and/or physician). In some embodiments, data collection system 1702 may include a light source, a detector, and/or other optical components for collecting data from a sample obtained from a subject.

In some embodiments, communication network 1706 can be any suitable communication network or combination of communication networks. For example, communication network 1706 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, etc.), a wired network, etc. In some embodiments, communication network 1706 can be a local area network, a wide area network, a public network (e.g., the Internet), a private or semi-private network (e.g., a corporate or university intranet), any other suitable type of network, or any suitable combination of networks. Communications links shown in FIG. 17 can each be any suitable communications link or combination of communications links, such as wired links, fiber optic links, Wi-Fi links, Bluetooth links, cellular links, etc.

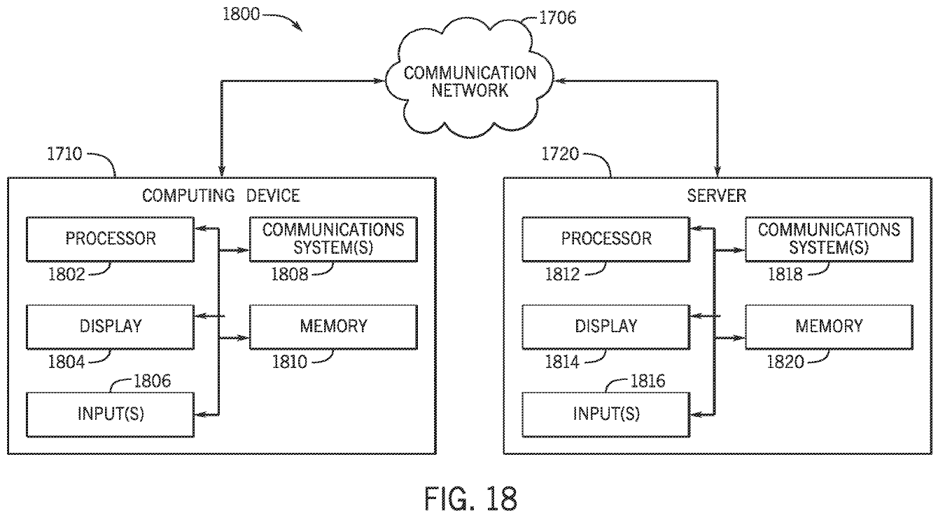

FIG. 18 shows an example 1800 of hardware that can be used to implement computing device 1710 and server 1720 in accordance with some embodiments of the disclosed subject matter. As shown in FIG. 18, in some embodiments, computing device 1710 can include a processor 1802, a display 1804, one or more inputs 1806, one or more communication systems 1808, and/or memory 1810. In some embodiments, processor 1802 can be any suitable hardware processor or combination of processors, such as a central processing unit, a graphics processing unit, etc. In some embodiments, display 1804 can include any suitable display devices, such as a computer monitor, a touchscreen, a television, etc. In some embodiments, inputs 1806 can include any suitable input devices and/or sensors that can be used to receive user input, such as a keyboard, a mouse, a touchscreen, a microphone, etc.

In some embodiments, communications systems 1808 can include any suitable hardware, firmware, and/or software for communicating information over communication network 1706 and/or any other suitable communication networks. For example, communications systems 1808 can include one or more transceivers, one or more communication chips and/or chip sets, etc. In a more particular example, communications systems 1808 can include hardware, firmware and/or software that can be used to establish a Wi-Fi connection, a Bluetooth connection, a cellular connection, an Ethernet connection, etc.

In some embodiments, memory 1810 can include any suitable storage device or devices that can be used to store instructions, values, etc., that can be used, for example, by processor 1802 to present content using display 1804, to communicate with server 1720 via communications system(s) 1808, etc. Memory 1810 can include any suitable volatile memory, non-volatile memory, storage, or any suitable combination thereof. For example, memory 1810 can include RAM, ROM, EEPROM, one or more flash drives, one or more hard disks, one or more solid state drives, one or more optical drives, etc. In some embodiments, memory 1810 can have encoded thereon a computer program for controlling operation of computing device 1710. In such embodiments, processor 1802 can execute at least a portion of the computer program to present content (e.g., images, user interfaces, graphics, tables, etc.), receive content from server 1720, transmit information to server 1720, etc.

In some embodiments, server 1720 can include a processor 1812, a display 1814, one or more inputs 1816, one or more communications systems 1818, and/or memory 1820. In some embodiments, processor 1812 can be any suitable hardware processor or combination of processors, such as a central processing unit, a graphics processing unit, etc. In some embodiments, display 1814 can include any suitable display devices, such as a computer monitor, a touchscreen, a television, etc. In some embodiments, inputs 1816 can include any suitable input devices and/or sensors that can be used to receive user input, such as a keyboard, a mouse, a touchscreen, a microphone, etc.

In some embodiments, communications systems 1818 can include any suitable hardware, firmware, and/or software for communicating information over communication network 1706 and/or any other suitable communication networks. For example, communications systems 1818 can include one or more transceivers, one or more communication chips and/or chip sets, etc. In a more particular example, communications systems 1818 can include hardware, firmware and/or software that can be used to establish a Wi-Fi connection, a Bluetooth connection, a cellular connection, an Ethernet connection, etc.

In some embodiments, memory 1820 can include any suitable storage device or devices that can be used to store instructions, values, etc., that can be used, for example, by processor 1812 to present content using display 1814, to communicate with one or more computing devices 1710, etc. Memory 1820 can include any suitable volatile memory, non-volatile memory, storage, or any suitable combination thereof. For example, memory 1820 can include RAM, ROM, EEPROM, one or more flash drives, one or more hard disks, one or more solid state drives, one or more optical drives, etc. In some embodiments, memory 1820 can have encoded thereon a server program for controlling operation of server 1720. In such embodiments, processor 1812 can execute at least a portion of the server program to transmit information and/or content (e.g., results of a tissue identification and/or classification, a user interface, etc.) to one or more computing devices 1710, receive information and/or content from one or more computing devices 1710, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

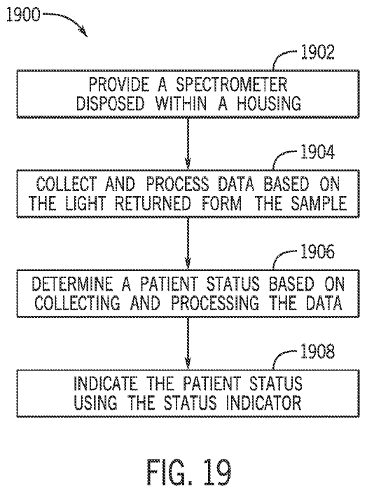

FIG. 19 shows an example 1900 of a process for biofluid monitoring in accordance with some embodiments of the disclosed subject matter. As shown in FIG. 19, at 1902, process 1900 can provide a spectrometer disposed within a housing, where the spectrometer may include a light source to illuminate a sample within a catheter tubing, a detector to detect light returned from the sample, a status signal indicator to provide patient status based on the sample in the catheter tubing, and a controller in communication with the light source, the detector, and the status signal indicator. At 1904, process 1900 can collect and process data based on the light returned from the sample, where the collecting and processing may be carried out by the controller. At 1906, process 1900 can determine a patient status based on collecting and processing the data, where determining may be carried out by the controller. Finally, at 1908, process 1900 can indicate the patient status using the status indicator, where indicating may be carried out by the controller. In various embodiments, the housing may be configured to attach at a low point in the catheter tubing such that the sample accumulates in the low point and the light source and the detector may be directed towards the low point to obtain the data from the sample.

It should be understood that the above described steps of the process of FIG. 19 can be executed or performed in any order or sequence not limited to the order and sequence shown and described in the figures. Also, some of the above steps of the processes of FIG. 19 can be executed or performed substantially simultaneously where appropriate or in parallel to reduce latency and processing times.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. A biofluid monitoring apparatus, comprising:
a spectrometer disposed within a housing, the spectrometer including:
a light source to illuminate a sample within a catheter tubing,
a detector to detect light returned from the sample,
a status signal indicator to provide patient status based on the sample in the catheter tubing, and
a controller in communication with the light source, the detector, and the status signal indicator to collect and process data based on the light returned from the sample to determine a patient status and indicate the patient status using the status indicator,
wherein the housing is configured to attach at a low point in the catheter tubing such that the sample accumulates in the low point, and
wherein the light source and the detector are directed towards the k point obtain the data from the sample.

2. The apparatus of claim 1, wherein the spectrometer further comprises a power supply.

3. The apparatus of claim 2, wherein the power supply comprises a battery.

4. The apparatus of claim 1, wherein the housing comprises a slot into which the catheter tubing is inserted such that a portion of the catheter tubing is adjacent to the spectrometer.

5. The apparatus of claim 1, wherein the spectrometer further comprises a collimator to focus light from the light source into the sample.

6. The apparatus of claim 5, wherein the collimator comprises a lens.

7. The apparatus of claim 1, wherein the spectrometer further comprises a monochromator to divide the light from the light source into a plurality of constituent wavelengths.

8. The apparatus of claim 7, wherein the monochromator comprises a prism.

9. The apparatus of claim 8, wherein the spectrometer further comprises a wavelength selector to select a particular wavelength to direct to the sample, wherein the particular wavelength is selected based on at least one of a bacterial strain or a bacterial product to be identified.

10. The apparatus of claim 9, wherein the wavelength selector comprises a slit.

11. The apparatus of claim 1, wherein the detector comprises a photocell to record one or more wavelengths of light returned from the sample based on the illumination of the sample.

12. The apparatus of claim 11, wherein the light returned from the sample measured by the detector comprises absorbance information.

13. The apparatus of claim 1, wherein the spectrometer further comprises a communication module to transmit information from the spectrometer.

14. The apparatus of claim 13, wherein the communication module comprises a radio communication device including at least one of a Bluetooth device, a cellular service device, or a WiFi device for performing wireless transmission.

15. The apparatus of claim 14, wherein the radio communication device including at least one of a Bluetooth device, cellular service device, or WiFi device performs wireless transmission to a computing platform comprising at least one of an electronic health record or a mobile computing device.

16. The apparatus of claim 15, wherein the mobile computing device comprises at least one of a cell phone, a smart phone, a pager, or a telephone.

17. The apparatus of claim 16, wherein the information from the spectrometer is transmitted as at least one of a text message, an audio message, an email, or a data file.

18. The apparatus of claim 1, wherein the controller determines the patient status using one or more machine learning algorithms specifically trained for the apparatus.

19. The apparatus of claim 18, wherein the one or more machine learning algorithms identify one or more biomarkers indicative of a functional status of a bodily system of the patient.

20. The apparatus of claim 19, wherein the bodily system of the patient comprises at least one of a cardiac system, a respiratory system, a renal system, a neurologic system, an endocrine system, or an immune system.

21. The apparatus of claim 20, wherein the one or more machine learning algorithms identifies at least one condition comprising at least one of: a bacterial colony count, a bacterial colony type, or a bacterial infection by-product.

22. The apparatus of claim 21, wherein the patient status is determined based on the identified at least one condition.

23. The apparatus of claim 1, wherein the status indicator is configured to indicate at least one of a plurality of states of the patient status.

24. The apparatus of claim 23, wherein the states of the patient status comprise at least one of: no bacteria or infection in the sample, bacterial colonization but no infection in the sample, or bacteria and infection in the sample.

25. The apparatus of claim 24, wherein the status indicator indicates the patient status using at least one light coupled to the housing.

26. The apparatus of claim 1, wherein the low point in the catheter tubing comprises a bend in the catheter tubing.

27. The apparatus of claim 26, wherein the housing comprises a curved face, and
wherein the bend in the catheter tubing is located adjacent to the curved face of the housing.

28. The apparatus of claim 1, further comprising a load cell sensor coupled to the housing, wherein the load cell sensor is coupled to a biofluid collection container fluidly coupled to the catheter tubing,
wherein the controller is coupled to the load cell sensor and configured to:
obtain data from the load cell sensor,
calculate a weight change of the biofluid collection container based on the data obtained from the load cell sensor, and
determine a flow rate of the sample into the biofluid collection contained based on the calculated weight change.

29. A method for biofluid monitoring, comprising:
providing a spectrometer disposed within a housing, the spectrometer including:
a light source to illuminate a sample within a catheter tubing,
a detector to detect light returned from the sample,
a status signal indicator to provide patient status based on the sample in the catheter tubing, and
a controller in communication with the light source, the detector, and the status signal indicator;
collecting and processing, using the controller, data based on the light returned from the sample;
determining, using the controller and based on collecting and processing the data, a patient status; and
indicating, using the controller, the patient status using the status indicator,
wherein the housing is configured to attach at a low point in the catheter tubing such that the sample accumulates in the low point, and
wherein the light source and the detector are directed towards the low point to obtain the data from the sample.

30. The method of claim 29, wherein the spectrometer further comprises a power supply.

31. The method of claim 30, wherein the power supply comprises a battery.

32. The method of claim 29, wherein the housing comprises a slot into which the catheter tubing is inserted such that a portion of the catheter tubing is adjacent to the spectrometer.

33. The method of claim 29, wherein the spectrometer further comprises a collimator, the method further comprising:
focusing light from the light source into the sample using the collimator.

34. The method of claim 33, wherein the collimator comprises a lens.

35. The method of claim 29, wherein the spectrometer further comprises a monochromator, the method further comprising:
dividing the light from the light source into a plurality of constituent wavelengths using the monochromator.

36. The method of claim 35, wherein the monochromator comprises a prism.

37. The method of claim 36, wherein the spectrometer further comprises a wavelength selector, the method further comprising:
selecting a particular wavelength to direct to the sample using the wavelength selector, wherein the particular wavelength is selected based on at least one of a bacterial strain or a bacterial product to be identified.

38. The method of claim 37, wherein the wavelength selector comprises a slit.

39. The method of claim 29, wherein the detector comprises a photocell, the method further comprising:
recording one or more wavelengths of light returned from the sample based on the illumination of the sample using the photocell.

40. The method of claim 39, wherein the light returned from the sample measured by the detector comprises absorbance information.

41. The method of claim 29, wherein the spectrometer further comprises a communication module, the method further comprising:
transmitting information from the spectrometer using the communication module.

42. The method of claim 41, wherein the communication module comprises a radio communication device including at least one of a Bluetooth device, a cellular service device, or a WiFi device, wherein transmitting information from the spectrometer using the communication module further comprises:
transmitting information wirelessly from the spectrometer using the radio communication device including at least one of a Bluetooth device, cellular service device, or WiFi device.

43. The method of claim 42, wherein the radio communication device including at least one of a Bluetooth device, cellular service device, or WiFi device performs wireless transmission to a computing platform comprising at least one of an electronic health record or a mobile computing device.

44. The method of claim 43, wherein the mobile computing device comprises at least one of a cell phone, a smart phone, a pager, or a telephone.

45. The method of claim 44, wherein the information from the spectrometer is transmitted as at least one of a text message, an audio message, an email, or a data file.

46. The method of claim 29, wherein determining the patient status further comprises:
determining the patient status using one or more machine learning algorithms specifically trained for the apparatus.

47. The method of claim 46, wherein determining the patient status using one or more machine learning algorithms specifically trained for the apparatus further comprises:
identifying one or more biomarkers indicative of a functional status of a bodily system of the patient using the one or more machine learning algorithms.

48. The method of claim 47, wherein the bodily system of the patient comprises at least one of a cardiac system, a respiratory system, a renal system, a neurologic system, an endocrine system, or an immune system.

49. The method of claim 48, wherein the one or more machine learning algorithms identifies at least one condition comprising at least one of: a bacterial colony count, a bacterial colony type, or a bacterial infection by-product.

50. The method of claim 49, wherein determining the patient status using one or more machine learning algorithms further comprises:
determining the patient status based on the identified at least one condition.

51. The method of claim 29, wherein indicating the patient status using the status indicator further comprises:
indicating at least one of a plurality of states of the patient status.

52. The method of claim 51, wherein the states of the patient status comprise at least one of: no bacteria or infection in the sample, bacterial colonization but no infection in the sample, or bacteria and infection in the sample.

53. The method of claim 52, wherein indicating the patient status using the status indicator further comprises:
indicating the patient status using at least one light coupled to the housing.

54. The method of claim 29, wherein the low point in the catheter tubing comprises a bend in the catheter tubing.

55. The method of claim 54, wherein the housing comprises a curved face, and
wherein the bend in the catheter tubing is located adjacent to the curved face of the housing.

56. The method of claim 29, wherein the housing comprises a load cell sensor coupled thereto, wherein the load cell sensor is coupled to a biofluid collection container fluidly coupled to the catheter tubing, and
wherein the method further comprises:
obtaining data from the load cell sensor,
calculating a weight change of the biofluid collection container based on obtaining the data from the load cell sensor, and
determining a flow rate of the sample into the biofluid collection contained based on calculating the weight change.
